(19) United States
(12) Patent Application Publication
(10) Pub. No.: US 2005/0245601 A1
(43) Pub. Date: Nov. 3, 2005

(54) Treatment of diseases involving ErbB2 kinase overexpression

(75) Inventor: Harold H. Schmitz, Bethesda, MD (US)

Correspondence Address: NADA JAIN, P.C., 560 White Plains Road, Suite 460, Tarrytown, NY, 10591, US

(73) Assignee: Mars, Incorporated, McLean, VA (US)

(21) Appl. No.: 10/961,992

(22) Filed: Oct. 8, 2004

Related U.S. Application Data
(60) Provisional application No. 60/510,604, filed on Oct. 10, 2003.

(51) Int. Cl. A61K 31/353

(52) U.S. Cl. 514/456

(57) ABSTRACT

This invention relates to compositions containing polyphenols such as flavanols and their related oligomers, and methods for treating conditions associated with ErbB2 kinase overexpression, for example, treatment of cancers involving ErbB2 kinase overexpression.

This application claims the benefit, under 35 USC Section 119, of the U.S. Provisional Appl. No. 60/510,604 filed Oct. 10, 2003, the disclosure of which is hereby incorporated herein by reference.

### FIELD OF THE INVENTION

This invention relates to compositions, and methods of use thereof, containing polyphenols such as flavanols and their related oligomers (procyanidins), for example, cocoa polyphenols such as cocoa flavanols and procyanidins for treating diseases involving ErbB2 kinase overexpression, such as cancers involving ErbB2 kinase overexpression.

### BACKGROUND OF THE INVENTION

Kinase designated ErbB2 belong to the Erb-B family of receptor tyrosine kinases, which includes four known members: EGFR (epidermal growth factor receptor, also referred to as ErbB1), ErbB2, ErbB3 and ErbB4. All are essential for normal development and functioning of normal cells, however, they are deregulated, particularly EGFR and ErbB2, in certain cancer patients (Anderson and Ahmad, Front Biosci., 2002, 7:d1926-40). ErbB2 has also been referred to as HER2 (human epidermal growth factor receptor 2) and NEU (see e.g. OMIM database, available at www.ncbi.nlm.nih.gov/entrez).

ErbB2 plays an important role in regulation of angiogenesis. Regulation of angiogenesis by the ErbB receptors is mediated by the formation of heterodimers containing ErbB2 in combination with the other members of ErbB family. Thus, inhibiting the ErbB2 gene would decrease the possibility of forming the more efficient ErbB2 heterodimers and decrease downstream signaling events, which lead to more angiogenic stimuli. ErbB receptors are coupled to several kinase signaling pathways, including ERK1/2 (p44/p42) MAPKs, phospholipase C, phosphatidylinositol 3-kinase, and c-Jun NH2-terminal kinases. ErbB2 signaling is also associated with PI-3/Akt kinase pathways, and with the polyamine synthesis pathway.

Overexpression of ErbB2 kinase was observed in certain subpopulations of patients suffering from breast, ovarian, prostate, lung, gastric, bladder, salivary gland, endometrial, pancreatic and epithelial cancers, non-small-lung cell carcinoma and laryngeal carcinoma (Dowsett et al., Eur. J. Cancer, 36:170-6, 2000; Wang and Hung, Semin. Oncol., 28:115-24, 2001; Eccles, Recent Res. Cancer Res., 157:41-54, 2000; Kazkayasi et al., Eur. Arch. Otorhinolaryngol., 258:329-35, 2001; Scholl et al., Ann. Oncol., 12 Suppl. 1:S81-7, 2001). For example, ErbB2 is overexpressed (usually as a result of proto-oncogene amplification) in 20-30% of breast cancers (Dowsett et al., Eur. J. Cancer, 36:170-6, 2000). Patients with breast cancer that overexpresses ErbB2 have a poor prognosis, short relapse time, higher rate of relapse, shorter survival time and low survival rate (Wang and Hung, Semin. Oncol., 28:115-24, 2001). Overexpression is associated with increased metastatic potential and resistance to chemotherapeutic agents. In breast cancers, overexpression of ErbB2 correlates with lymph node metastasis. Therefore there is a need in the art to design therapies targeted specifically to treatment of the patient subpopulations where the aggressiveness of the tumor is increased.

Presently, ErbB2 kinase is a target for anti-ErbB2 monoclonal antibody therapy (Slamon et al., N. Engl. J. Med., 344:783-92, 2001). Given the gravity of the diseases involving ErbB2 overexpression, additional approaches and agents for treatment are needed. Applicants have now unexpectedly discovered that certain polyphenols, such as flavanols and their related oligomers, including those obtained from cocoa, are effective at reducing ErbB2 overexpression and are useful for the treatment of diseases such as cancers that overexpress ErbB2.

### SUMMARY OF THE INVENTION

The invention relates to compositions, products and methods for treatment or prevention of conditions characterized by ErbB2 overexpression, for example treatment and chemoprevention of cancers that overexpress ErbB2 kinase.

In one aspect, the invention relates to a composition, such as a food, a food additive, a dietary supplement, or a pharmaceutical comprising a polyphenol, such as a flavanol and/or its related oligomer. The composition may optionally contain an additional cancer treating agent.

Packaged products containing the above-mentioned compositions and a label and/or instructions for use to treat cancer that overexpresses ErbB2 kinase are also within the scope of the invention.

In another aspect, the invention relates to a method for treating cancer that overexpresses ErbB2 kinase in a mammal, such as a human or a veterinary animal, by administering a composition containing a polyphenol such as a flavanol and/or its related oligomers to the mammal in need thereof.

In yet another aspect, the invention relates to methods of chemoprevention of cancer that overexpresses ErbB2 kinase in a mammal, such as a human or a veterinary animal, by administering a composition containing a polyphenol such as a flavanol and/or its related oligomers to the mammal in need thereof.

### BRIEF DESCRIPTION OF THE DRAWINGS

FIG. 1 represents an autoradiograph of tyrosine kinase PCR fragments prepared from HAEC (Human Aortic Endothelial Cells) treated with procyanidins and digested with RsaI restriction enzyme. Gel bands showing differentially expressed ErbB2 kinase are marked. Lanes: M=monomer, D=dimer, P=pentamer, O=octamer, E=epicatechin, C=control saline

FIG. 2 represents results from the real time PCR experiments verifying reduced expression of ErbB2 kinase in HAEC in the presence of procyanidins.

FIG. 3 represents cell proliferation experiment conducted in the presence of a procyanidin monomer or pentamer, with and without the addition of Vascular Endothelial Growth Factor (VEGF).

FIG. 4A represents an autoradiograph of tyrosine kinase PCR fragments prepared from micro dermal endothelial cells treated with procyanidins and digested with RsaI restriction enzyme. Digest bands representing tyrosine kinases MET and ErbB2, and the size of the digest products, are shown. Lanes: M=monomer, P=pentamer, C=control saline.

FIG. 4B represent an autoradiograph of tyrosine kinase PCR fragments prepared from micro dermal endothelial cells treated with procyanidins and digested with Bsp1286I. Digest bands representing tyrosine kinases ErbB2 and VEGFR-3, and the size of the digest products, are shown. Lanes: M=monomer, P=pentamer, C=control saline.

FIG. 5 represents quantitative real time PCR experiments verifying reduced ErbB2, KDR/VEGFR-2 and MAPK11 gene expression (20 μg/ml pentamer-pretreated and control HMDEC).

FIG. 6 represents a Western blot of pentamer-pretreated and control HMDEC lysate probed with RC20H antibody

FIG. 7 represents a Western blot of pentamer-pretreated and control HMDEC exposed to 1 mM H₂O₂ for 30 minutes and probed with recombinant HRP-Anti-PhosphoTyrosine (PY20) antibody.

FIG. 8 represents proliferation of pentamer-treated and octamer-treated HMDEC stimulated with 1 mM H₂O₂ for 30 minutes. Agst refers to angiogenic simulation (1 mM H₂O₂).

### DETAILED DESCRIPTION

All patents, patent applications and references cited in this application are hereby incorporated herein by reference. In case of any inconsistency, the present disclosure governs.

The present invention relates to a composition containing a flavanol and/or its related oligomer, such as at least one flavanol and/or its related procyanidin oligomer, and the administration of the composition to a mammal in need thereof. For example, the composition may comprise an effective amount of the flavanol having the following formula, or a pharmaceutically acceptable salt, derivative or oxidation product thereof: 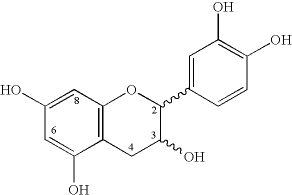
Alternatively, or in combination, the composition may comprise an effective amount of the polymeric compound of the formula Aₙ or a pharmaceutically acceptable salt, derivative or oxidation product thereof: 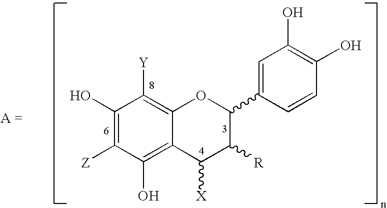
wherein
n is an integer from 2 to 18;
R and X each have either α or β stereochemistry;
R is OH;
the substituents of C-4, C-6 and C-8 are X, Z and Y, respectively, and bonding of monomeric units occurs at C-4, C-6 and C-8; and
when any C-4, C-6 or C-8 are not bonded to another monomeric unit, X, Y and Z are hydrogen.

In one embodiment the polymeric compound Aₙ has the following formula: 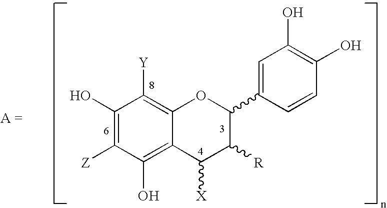
wherein
n is 5;
R and X each have either α or β stereochemistry;
R is OH;
the substituents of C-4, C-6 and C-8 are X, Z and Y, respectively, and bonding of monomeric units occurs at C-4, C-6 and C-8; and
when any C-4, C-6 or C-8 are not bonded to another monomeric unit, X, Y and Z are hydrogen;
or a pharmaceutically acceptable salts, derivative or oxidation product thereof.

Examples of the pentamers may be: [EC-(4β→8)]₄-EC, [EC-(4β→8)]₃-EC-(4β→6)-EC, [EC-(4β→8)]₃-EC-(4β→8)-C, and [EC-(4β→8)]₃-EC-(4β→6)-C, wherein EC is epicatechin and C is catechin. Both purified individual pentamers and pentamer mixtures may be used. The degree of purity may be, for example, at least about 50%, or at least about 60%, or at least about 70%, or at least about 80%, or at least about 90%, or at least about 92%, or at least about 95%, or at least about 98%, or at least about 99%.

In another embodiment, the polymeric compound Aₙ has the following formula: 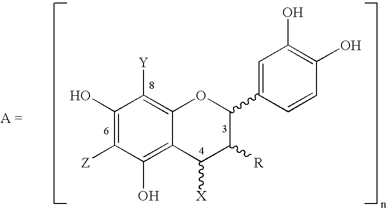
wherein
n is 8;
R and X each have either α or β stereochemistry;
R is OH;
the substituents of C-4, C-6 and C-8 are X, Z and Y, respectively, and bonding of monomeric units occurs at C-4, C-6 and C-8; and
when any C-4, C-6 or C-8 are not bonded to another monomeric unit, X, Y and Z are hydrogen;
or a pharmaceutically acceptable salts, derivative or oxidation product thereof.

Examples of the octamers may be: [EC-(4β→8)]₇-EC, [EC-(4β→8)]₆-EC-(4β→6)-EC, [EC-(4β→8)]₆-EC-(4β→8)-C, and [EC-(4β→8)]₆-EC-(4β→6)-C, wherein EC is epicatechin and C is catechin. Both purified individual octamers and octamer mixtures may be used. The degree of purity may be, for example, at least about 50%, or at least about 60%, or at least about 70%, or at least about 80%, or at least about 90%, or at least about 92%, or at least about 95%, or at least about 98%, or at least about 99%.

The flavanol and/or its related oligomer may be a cocoa flavanol and/or a cocoa procyanidin oligomer. In addition to, or in place of, the cocoa polyphenols, compositions may contain polyphenols from sources other than cocoa, which have structures and/or properties same or similar to those of cocoa polyphenols. The composition may optionally contain an additional cancer treating agent.

As used herein, the term “cocoa polyphenol” (CP) refers to polyphenolic substances such as flavanols and their related oligomers which are characteristic of cocoa beans. In other words, a cocoa polyphenol, a cocoa flavanol or a cocoa procyanidin oligomer is any such polyphenol, flavanol or procyanidin oligomer, irrespective of its source, which has a structural formula of the polyphenol, flavanol or procyanidin naturally occurring in cocoa. In one embodiment, these compounds may be extracted from cocoa beans or cocoa ingredients. The term “flavanol” includes the monomers catechin and epicatechin. Oligomers of catechin and epicatechin are referred to as procyanidins. Any reference to polyphenol herein should be understood to also apply to flavanols and procyanidin, in combination and individually, and vice versa.

The term “cocoa ingredient” refers to a cocoa solids-containing material derived from shell-free cocoa nibs such as chocolate liquor and partially or fully-defatted cocoa solids (e.g. cake or powder).

The polyphenols for use in the present invention may be of natural origin, for example, derived from a cocoa bean or another natural source of polyphenols, or prepared synthetically. A person of skill in the art may select natural or synthetic polyphenol based on availability or cost. Polyphenols may be included in the composition in the form of a cocoa ingredient containing cocoa polyphenols, for example, chocolate liquor included in chocolate, or may be added independently of cocoa ingredients, for example, as an extract, extract fraction, isolated and purified individual compound, pooled extract fractions or a synthetically prepared compound.

Flavanols include (+)-catechin, (−)-epicatechin and their respective epimers (e.g. (−)-catechin and (+)-epicatechin) and have the structure:

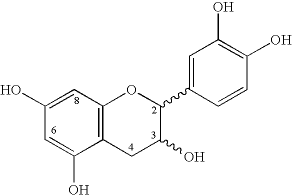

The procyanidin oligomers may have from 2 to about 18, preferably from 2 to about 12, and most preferably from 2 to about 10 monomeric units. Alternatively, the oligomers may have from 3-18, preferably 3-12, and more preferably 3-10 monomeric units; or from 5-18, preferably 5-12 and more preferably 5-10 monomeric units. For example, oligomers may be dimers, trimers, tetramers, pentamers, hexamers, heptamers, octamers, nonamers and decamers. In the oligomer, monomers are connected via interflavan linkages of (4→6) and/or (4→8). Oligomers with exclusively (4→8) linkages are linear; while the presence of at least one (4→6) bond results in a branched oligomer. Also within the scope of the invention are oligomers comprising at least one non-natural linkage (6→6), (6→8), and (8→8). The synthesis of such non-naturally occurring oligomers is described in the International Appl. No. PCT/US00/08234 published on Oct. 19, 2000 as WO 00/61547, the relevant portions of which are hereby incorporated herein by reference.

The cocoa polyphenol may be prepared by extraction from cocoa beans, cocoa nibs, or cocoa ingredients such as chocolate liquor, partially defatted cocoa solids, and/or fully defatted cocoa solids. Preferably, the extract is prepared from a fully or partially defatted cocoa powder. Beans from any species of Theobroma, Herrania or inter- and intra-species crosses thereof may be used. The extract may be prepared from fermented, underfermented or unfermented beans, the fermented beans having the least amount of cocoa polyphenols and the unfermented the most. The selection of beans may be made based on the fermentation factor of the beans, for example, the extract may be made from the beans having a fermentation factor of about 275 or less. Optimizing the level of polyphenols in the cocoa ingredient and extract thereof by manipulating the degree of fermentation may be done as described in the International Appl. No. PCT/US97/15893 published as WO98/09533, the relevant portions of which are hereby incorporated herein by reference.

Cocoa polyphenols may be extracted from cocoa ingredients that have been processed using traditional methods of cocoa processing (described, for example, in Industrial Chocolate Manufacture and Use, ed. Beckett, S. T., Blackie Acad. & Professional, New York, 1997, such as in Chapters 1, 5 and 6) or using an improved processing method described in U.S. Pat. No. 6,015,913 to Kealey et al. that preserves polyphenols (by preventing their destruction) in cocoa ingredients in contrast to the traditional methods. The improved cocoa processing method omits the traditional roasting step. Thus, cocoa ingredients obtainable by (a) heating the cocoa bean for a time and a temperature sufficient to loosen the cocoa shell without roasting the cocoa nib; (b) winnowing the cocoa nib from the cocoa shell; (c) screw pressing the cocoa nib and (d) recovering the cocoa butter and partially defatted cocoa solids which contain preserved levels of cocoa polyphenols, may be used. The method retains a much higher level of higher procyanidin oligomers than traditional processing methods. Cocoa solids produced by this method may contain greater than 20,000 μg of total flavanol and/or procyanidins per gram nonfat solids; preferably greater than 25,000 μg/g, more preferably greater than 28,000 μg/g, and most preferably greater than 30,000 μg/g. For purposes of this invention, the total flavanol and/or procyanidin amounts are determined as described in Example 1.

Polyphenols may be extracted from the sources indicated above, or any other polyphenol or flavanol or procyanidin containing source, using solvents in which the polyphenols dissolve. Suitable solvents include water or organic solvent such as methanol, ethanol, acetone, isopropyl alcohol and ethyl acetate. Solvent mixtures may also be used. When water is used as the solvent, it may be slightly acidified, for example with acetic acid. Examples of some solvents are mixtures of water and organic solvent, for example aqueous methanol, ethanol or acetone. Aqueous organic solvents may contain, for example, from about 50% to about 95% of organic solvent. Thus, about 50%, about 60%, about 70%, about 80% and about 90% organic solvent in water may be used. The solvent may also contain a small amount of acid such as acetic acid, for example, in the amount of about 0.5% to about 1.0%. The composition of the extracts, i.e., the representation (i.e., oligomeric profile) and the amount of procyanidin oligomers, will depend on the choice of solvents. For example, the water extract contains primarily monomers, the ethyl acetate extract contains monomers and lower oligomers, mainly dimers and trimers, and the aqueous methanol, ethanol or acetone extract contains monomers and a range of higher oligomers. One of the solvents for extraction of monomer as well as higher procyanidin oligomers is about 70% acetone. However, any extract containing polyphenols is useful in the invention. The methods of cocoa polyphenol extraction are known in the art and are described, for example, in the U.S. Pat. No. 5,554,645 to Romanczyk et al. and the International Appl. No. PCT/US97/05693, published as WO97/36497. Thus, in one embodiment, the cocoa extract is prepared by reducing cocoa beans to cocoa powder, defatting the powder, extracting the cocoa polyphenols, and purifying the extract. The cocoa powder can be prepared by freeze-drying the cocoa beans and pulp, depulping and dehulling the freeze-dried cocoa beans, and grinding the dehulled beans.

The cocoa polyphenol extract may be purified, for example, by removal of the caffeine and/or theobromine, and further purified by gel permeation chromatography and/or High Pressure Liquid Chromatography (HPLC). Gel permeation chromatography (e.g. on Sephadex LH-20) may be used to enrich the extract for higher procyanidin oligomers. For example, the eluate containing monomers and lower oligomers may not be collected until the oligomer(s) of choice begins eluting from the column. An example of such an extract is known in the art and is described in Example 5 of the International Appl. No. PCT/US97/05693, published as WO97/36497, the relevant portions of which are hereby incorporated by reference herein. By using preparative HPLC, for example, normal phase HPLC, the extract may be fractionated, for example, into monomeric and oligomeric fractions containing at least 50% by weight of the monomer or specific oligomer(s). When a particular fraction contains the monomers or any of the lower oligomers (e.g. dimers, trimers or tetramers fraction), the fraction contain about 90 to 95% by weight of the particular oligomeric fraction. The desired fractions may be pooled after separation to obtain a combination of oligomers of choice for example to contain oligomers 3-10 or 5-10. A person of skill in the art can manipulate the chromatographic conditions to achieve the desired procyanidin profile in view of the guidance in this specification, general knowledge in the art and, for example, the teachings of U.S. Pat. No. 5,554,645 to Romanczyk et al. and the International Appl. No. PCT/US97/05693, published as WO97/36497.

The monomeric fraction typically contains a mixture of monomers epicatechin and catechin; and the oligomeric fraction typically contains a mixture of dimers (in a dimer fraction), trimers (in a trimer fraction), tetramers (in a tetramer fraction), etc. Mixtures of monomers and oligomers occur in isolated fractions because cocoa contains more than one type of each of monomer, dimer, etc. The oligomeric variability occurs as a result of two monomers, epicatechin and catechin, that are building blocks of procyanidins, as well as the chemical bond connecting monomers in the oligomer. Thus, cocoa dimers are primarily B2 and B5, each of which contains two monomers of epicatechin. Individual monomers and oligomers may be obtained using reversed-phase HPLC, e.g. using a C18 column.

Cocoa polyphenol may be used in the compositions of the invention as a cocoa extract, e.g. solvent-derived extract, cocoa fraction, isolated compounds or in the form of a cocoa ingredient or a chocolate containing an effective amount of cocoa flavanols and/or procyanidins. The cocoa ingredients may be prepared using traditional cocoa processing procedures but is preferably prepared using the method described in U.S. Pat. No. 6,015,913 to Kealey et al. Alternatively, to enhance the level of cocoa polyphenols, chocolate liquor and cocoa solids prepared from cocoa beans having a fermentation factor of about 275 or less may be used. These ingredients have cocoa polyphenol content that is higher than can be obtained using traditional cocoa processing methods (e.g. with roasting) and fully fermented beans. The chocolate may be prepared using conventional techniques from the ingredients described above or using an improved process for preserving cocoa polyphenols during chocolate manufacturing as described in the International Appl. No. PCT/US99/05414 published as WO99/45788, the relevant portions of which are hereby incorporated herein by reference. A chocolate prepared by at least one of the following non-traditional processes is referred to herein as a “chocolate having a conserved amount of cocoa polyphenols”: (i) preparing cocoa ingredients from underfermented or unfermented cocoa beans; (ii) preserving cocoa polyphenol during cocoa ingredient manufacturing process; and (iii) preserving cocoa polyphenol during chocolate manufacturing process.

In some embodiments, the compositions contain at least one oligomer, such as for example a dimer. Such compositions may additionally contain at least one monomer or a combination of monomers, e.g. catechin and epicatechin. In another embodiment, compositions containing a combination of monomers, catechin and epicatechin, e.g. in a form of a monomer fraction isolated from cocoa, are also prepared and used.

Synthetic procyanidins may also be used and are prepared by methods known in the art and as described for example in the International Appl. No. PCT/US98/21392 published as WO99/19319, the relevant portions of which are hereby incorporated herein by reference.

Flavanol and/or procyanidin derivatives may also be useful. These include esters of monomer and oligomers such as the gallate esters (e.g. epicatechin gallate and catechin gallate); compounds derivatized with a saccharide moiety such as mono- or di-saccharide moiety (e.g. β-D-glucose), for example at positions X, Y and/or Z in the above formulas; glycosylated monomers and oligomers, and mixtures thereof; metabolites of the procyanidin monomers and oligomers, such as the sulphated, glucoronidated, and methylated forms except for the enzyme cleavage products of procyanidins generated by colonic microflora metabolism. The derivatives may be from natural sources or prepared synthetically.

The composition of the invention is useful as a pharmaceutical, a food, a food additive, a dietary supplement, or a pharmaceutical. The compositions may contain a carrier, a diluent, or an excipient. Depending on the intended use, the carrier, diluent, or excipient may be chosen to be suitable for human or veterinary use, food, additive, supplement or pharmaceutical use.

As used herein a “food” is a material consisting essentially of protein, carbohydrate and/or fat, which is used in the body of an organism to sustain growth, repair and vital processes and to furnish energy. Foods may also contain supplementary substances such as minerals, vitamins and condiments. See Merriam-Webster's Collegiate Dictionary, 10th Edition, 1993. The term food includes a beverage adapted for human or animal consumption. As used herein a “food additive” is as defined by the FDA in 21 C.F.R. 170.3(e)(1) and includes direct and indirect additives. As used herein, a “pharmaceutical” is a medicinal drug. See Merriam-Webster's Collegiate Dictionary, 10th Edition, 1993. A pharmaceutical may also be referred to as a medicament. As used herein, a “dietary supplement” is a product (other than tobacco) that is intended to supplement the diet that bears or contains the one or more of the following dietary ingredients: a vitamin, a mineral, an herb or other botanical, an amino acid, a dietary substance for use by man to supplement the diet by increasing the total daily intake, or a concentrate, metabolite, constituent, extract or combination of these ingredients.

Any conventional food including any beverage which has been improved by the presence of a polyphenol or a derivative thereof, e.g. methylated compounds or metabolic breakdown products, and optionally in combination with another cancer treating agent. Other compounds, such as L-arginine, calcium, potassium, magnesium, an oxidant such as vitamin E and vitamin C, any of the vitamins of the B complex, a carotenoid, guar gum, and/or a mono or polyunsaturated fatty acid (e.g. omega-3), may also be present.

The improvement is achieved either (i) by adding polyphenol or a derivative thereof to a food that does not contain cocoa polyphenol or (ii) when the food traditionally may contain cocoa polyphenols, such as for example chocolate, by enhancing the polyphenol level over the one found in the traditionally prepared food. The enhancement may be achieved by adding additional polyphenols such as cocoa polyphenols, for example, in a form of an extract, fraction or isolated and purified compound there from; by adding cocoa polyphenol in combination with another polyphenol containing ingredient (e.g. nut skins); by manipulating the cocoa ingredients processing and cocoa bean selection, as described above, to preserve cocoa polyphenol in the cocoa ingredient used for the manufacture of the food product; or by manipulating the chocolate manufacturing process as described above. Thus, these foods (including beverages) contain an “elevated level of polyphenols” (including cocoa procyanidins) in comparison to comparative conventional foods (including beverages). An example of a chocolate having an elevated level of polyphenol occurs when a chocolate manufacturer adds a cocoa extract containing cocoa polyphenols to its previously commercially available product. The foods may also be referred to as “high cocoa polyphenol foods,” i.e., they contain higher levels of polyphenol than their traditional counterparts.

The foods comprising cocoa polyphenols and optionally another cancer treating agent may be adapted for human or veterinary use, and include pet foods. The food may be other than a confectionery, however, the preferred cholesterol lowering food is a confectionery such as a standard of identity (SOI) and non-SOI chocolate, such as milk, sweet and semi-sweet chocolate including dark chocolate, low fat chocolate and a candy which may be a chocolate covered candy. Other examples include a baked product (e.g. brownie, baked snack, cookie, biscuit) a condiment, a granola bar, a toffee chew, a meal replacement bar, a spread, a syrup, a powder beverage mix, a cocoa or a chocolate flavored beverage, a pudding, a rice cake, a rice mix, a savory sauce and the like. If desired, the foods may be chocolate or cocoa flavored. Food products may be chocolates and candy bars, such as granola bars, containing nuts, for example, peanuts, walnuts, almonds, and hazelnuts. It should be noted that the addition of nuts with skins to the food described herein may also increase the total polyphenol content since, for example, peanut skins contain about 17% flavanols and procyanidins and almond skins contain about 30% flavanols and procyanidins. In one embodiment, the nut skins are added to the nougat of a chocolate candy.

In certain embodiments, the non-chocolate food product contains from about at least 5 micrograms/g to about 10 mg/g, and, for example, at least 5 micrograms/g food product, preferably at least 10 microgram/g, more preferably at least 100 micrograms/g of cocoa flavanols and/or procyanidin oligomers. If desired, the non-chocolate food products can contain much higher levels of cocoa procyanidins than those found in the chocolate food products described below.

In one embodiment, a chocolate confectionery contains an effective amount of cocoa flavanol and/or procyanidin to treat, prevent, reduce the risk of, or reduce the occurrences of heart arrhythmia, or generally any condition associated with abnormalities in gap junctional communication, such as for example, a neuro-degenerative disease. The chocolate confectionery may be milk or dark chocolate. In certain embodiments, the chocolate comprises at least 3,600 micrograms, preferably at least 4,000 micrograms, preferably at least 4,500 micrograms, more preferably at least 5,000 micrograms, and most preferably at least 5,500 micrograms cocoa flavanols and/or procyanidins each per gram of chocolate, based on the total amount of nonfat cocoa solids in the product. In other embodiments, the chocolate contains at least 6,000 micrograms, preferably at least 6,500 micrograms, more preferably at least 7,000 micrograms, and most preferably at least 8,000 micrograms of cocoa procyanidins per gram, and even more preferably 10,000 micrograms/g based on the nonfat cocoa solids in the product.

A milk chocolate confectionery may have at least 1,000 micrograms, preferably at least 1,250 micrograms, more preferably at least 1,500 micrograms, and most preferably at least 2,000 micrograms cocoa flavanols and/or procyanidins each per gram of milk chocolate, based on the total amount of nonfat cocoa solids in the milk chocolate product. In the preferred embodiment, the milk chocolate contains at least 2,500 micrograms, preferably at least 3,000 micrograms, more preferably at least 4,000 micrograms, and most preferably at least 5,000 micrograms cocoa flavanols and/or procyanidins each per gram of milk chocolate, based on the total amount of nonfat cocoa solids in the milk chocolate product.

The amount of L-arginine in the food products can vary. Typically, cocoa contains between 1 to 1.1 grams of L-arginine per 100 grams of partially defatted cocoa solids. It can range from 0.8 to 1.5 per 100 grams of cocoa. In some embodiments, the chocolate food products of this invention contain L-arginine in an amount greater than that which naturally occurs in the cocoa ingredients. Knowing the amount of cocoa ingredients and L-arginine used in the food product, one of ordinary skill in the art can readily determine the total amount of L-arginine in the final product. The food product will generally contain at least 5 micrograms/g, preferably at least 30 micrograms/g, or at least 60 micrograms/g, even more preferably at least 200 micrograms/g food product.

A daily effective amount of a polyphenol such as a flavanols and/or procyanidins may be provided in a single serving. Thus, a confectionery (e.g. chocolate) may contain at least about 100 mg/serving (e.g. 150-200, 200-400 mg/serving) cocoa procyanidins.

Pharmaceuticals containing flavanols and/or procyanidins, optionally in combination another cancer treating agent may be administered in a variety of ways such as orally, sublingually, bucally, nasally, rectally, intravenously, parenterally and topically. A person of skill in the art will be able to determine a suitable mode of administration to maximize the delivery of the compound of formula An, and optionally another cancer treating agent, to the site of the tumor. Thus, dosage forms adapted for each type of administration are within the scope of the invention and include solid, liquid and semi-solid dosage forms, such as tablets, capsules, gelatin capsules (gelcaps), bulk or unit dose powders or granules, emulsions, suspensions, pastes, creams, gels, foams or jellies. Sustained-release dosage forms are also within the scope of the invention and may be prepared as described in U.S. Pat. Nos. 5,024,843; 5,091,190; 5,082,668; 4,612,008 and 4,327,725, relevant portions of which are hereby incorporated herein by reference. Suitable pharmaceutically acceptable carriers, diluents, or excipients are generally known in the art and can be determined readily by a person skilled in the art. The tablet, for example, may comprise an effective amount of the polyphenol-containing composition and optionally a carrier, such as sorbitol, lactose, cellulose, or dicalcium phosphate.

The dietary supplement containing cocoa flavanol and/or procyanidin, and optionally another cancer treating agent, may be prepared using methods known in the art and may comprise, for example, nutrient such as dicalcium phosphate, magnesium stearate, calcium nitrate, vitamins, and minerals.

Further within the scope of the invention is a package comprising the composition of the invention (e.g. a food, a dietary supplement, a pharmaceutical) and a label indicating the presence of, or an enhanced content of flavanol and/or procyanidin and/or a derivative thereof, or directing use of the composition to treat cancer that overexpresses ErbB2. The package may contain the composition and the instructions for use to treat cancer or other condition characterized by overexpression of ErbB2.

As used herein, “treatment” means improving an existing medical condition, for example, cancer that overexpresses ErbB2, for example by slowing down the disease progression, prolonging survival, and reducing the risk of death. The term “preventing” means reducing the risks associated with developing a disease, including reducing the onset of the disease. The prevention or prophylaxis may be used in an individual known to be at high risk of developing cancer that overexpresses ErbB2. The methods may be used in a human or a veterinary animal, such as a dog, a cat, and a horse.

The methods comprise administering to a mammal, preferably a human or a veterinary animal, for an effective period of time (for example, to achieve the therapeutically relevant reduction of ErbB2 overexpression), an amount of the composition comprising a flavanol and/or its related oligomer, such as a cocoa flavanol and/or its related oligomer, optionally in combination with another cancer treating agent, the flavanol and/or its related oligomer being in the amount effective to treat cancer that overexpresses ErbB2.

In one embodiment, the invention relates to a method of treating and/or preventing cancer characterized by ErbB2 overexpression by administering, to a human or a veterinary animal, a composition comprising an effective amount of a pentamer or an octamer for an effective period of time.

Thus, the following use is within the scope of the invention. Use of a flavanol and/or its oligomer in the manufacture of a medicament, food, nutraceutical or dietary supplement for use of a condition characterized by ErbB2 overexpression. In one embodiment, use of a flavanol and/or its oligomer in the manufacture of a medicament, food, nutraceutical or dietary supplement for treating or reducing the risk of a cancer that overexpresses ErbB2 in a human or a veterinary animal is provided. The flavanol and/or its oligomer may be cocoa flavanol and/or cocoa flavanol oligomer.

The following uses are representative of some embodiments. Use of a flavanol and/or its oligomer in the manufacture of a medicament, food, nutraceutical or dietary supplement for use in treating, or reducing the risk of, a breast cancer that overexpresses ErbB2. Use of a flavanol and/or its oligomer in the manufacture of a medicament, food, nutraceutical or dietary supplement for use in treating, or reducing the risk of, ovarian, prostate, bladder, endothelial, salivary gland, endometrial, and pancreatic cancer, and laryngeal carcinoma and non-small-lung cell carcinoma that overexpresses ErbB2 in a human. Comparable cancers in a veterinary animal may also be treated.

The effective amount may be determined by a person of skill in the art using the guidance provided herein. For example, the effective amount may be such as to achieve a physiologically relevant concentration in the body of a mammal. Such a physiologically relevant concentration may be at least 20 nanomolar (nM), preferably at least about 100 nM, and more preferably at least about 500 nM. In one embodiment, at least about one micromole in the blood of the mammal is achieved.

The methods may further comprise determining the effectiveness of the treatment by, for example, measuring the level of ErbB2 expression or cell proliferation.

The composition may be administered to a healthy mammal for prophylactic purposes or to a mammal in need of a treatment or having at least one of the risk factors associated with cancer that overexpresses ErbB2. For example, the compositions may be administered to individuals with a familial history of such cancers. Other populations of mammals that are susceptible to developing cancer that overexpresses ErbB2 will be apparent to a person of skill in the art.

The effective amount to treat, or reduce the risk of, cancer that overexpresses ErbB2 may be determined by a person skilled in the art using the guidance provided herein and the general knowledge in the art. Flavanols and/or related oligomers may be administered at from about 50 mg/day to about 1000 mg/day, preferably from about 100-150 mg/day to about 900 mg/day, and most preferably from about 300 mg/day to about 500 mg/day. However, amounts higher than stated above may be used.

The treatments/preventive administration may be continued as a regimen, i.e., for an effective period of time, e.g., daily, monthly, bimonthly, biannually, annually, or in some other regimen, as determined by the skilled medical practitioner for such time as is necessary. The administration may be continued for at least a period of time required to reduce ErbB2 overexpression to a therapeutically relevant level of ErbB2 expression. Preferably, the composition is administered daily, most preferably two or three times a day, for example, morning and evening to maintain the levels of the effective compounds in the body of the mammal. To obtain the most beneficial results, the composition may be administered for at least about 30 to about 60 days. These regiments may be repeated periodically.

The invention is further described in the following non-limiting examples.

### EXAMPLES

### Example 1

### Determination of Flavanols/Procyanidins

Procyanidins were quantified as follows: a composite standard was made using commercially available (−)-epicatechin, and dimers through decamers obtained in a purified state by the methods described in Hammerstone, J. F. et al., J. Ag. Food Chem.; 1999; 47 (10) 490-496; Lazarus, S. A. et al., J. Ag. Food Chem.; 1999; 47 (9); 3693-3701; and Adamson, G. E. et al., J. Ag. Food Chem.; 1999; 47 (10) 4184-4188. Standard Stock solutions using these compounds were analyzed using the normal-phase HPLC method described in the previously cited Adamson reference, with fluorescence detection at excitation and emission wavelengths of 276 nm and 316 nm, respectively. Peaks were grouped and their areas summed to include contributions from all isomers within any one class of oligomers and calibration curves were generated using a quadratic fit. Monomers and smaller oligomers had almost linear plots which is consistent with prior usage of linear regression to generate monomer-based and dimer-based calibration curves.

These calibration curves were then used to calculate procyanidin levels in samples prepared as follows: First, the cocoa or chocolate sample (about 8 grams) was defatted using three hexane extractions (45 mL each). Next, one gram of defatted material was extracted with 5 mL of the acetone/water/acetic acid mixture (70:29.5:0.5 v/v). The quantity of procyanidins in the defatted material was then determined by comparing the HPLC data from the samples with the calibration curves obtained as described above (which used the purified oligomers). The percentage of fat for the samples (using a one gram sample size for chocolate or one-half gram sample size for liquors) was determined using a standardized method by the Association of Official Analytical Chemists (AOAC Official Method 920.177). The quantity of total procyanidin levels in the original sample (with fat) was then calculated. Calibration was performed prior to each sample run to protect against column-to-column variations.

### Example 2

### Kinase Profiling Expression Assay

The example shows that procyanidins down regulate expression of ErbB2 receptor kinase. As described in detail below, the showings were made using a kinase profiling expression assay and the results were verified by quantitative Real Time Polymerase Chain Reaction (PCR). Additionally, procyanidins suppressed growth of human aortic endothelial cells (HAEC) and human micro dermal endothelial cells (HMDEC), which inhibition of growth may in part be caused by ErbB2 down regulation and subsequent downsteam events. Mitogen-activated protein kinase 11 (MAPK11) and KDR were also down regulated.

Materials and Methods

Purification of Procyanidin Oligomers by Preparative Normal-Phase HPLC.

Approximately 0.7 g of semipurified acetone extract of cocoa was dissolved in 7 mL of acetone/water/acetic acid in a ratio by volume of 70:29.5:0.5, respectively. Separations were effected at ambient temperature using a 5 m Supelcosil LC-Si 100 Å. Procyanidins were eluted by a linear gradient. Separations of oligomers were monitored by UV at 280 nm, and fractions were collected at the valleys between the peaks corresponding to oligomers. Fractions with equal retention times from several preparative separations were combined, rotary evaporated under partial vacuum and freeze-dried. Monomer fractions included both epicatechin and catechin. Epicatechin was purchased from Sigma.

Cell Treatment

For the kinase profiling experiments, varying concentrations of procyanidins and controls (2.5-40 μg/ml) were incubated with 75% confluent human aortic endothelial cells (HAEC) in Medium 200 media (Cascade Biologics, Inc) containing 10% fetal calf serum and supplemented with 2% v/v hydrocortisone, 1 mg/ml human epidermal growth factor (EGF), 10 ng/ml basic fibroblast growth factor (bFGF), 3 ng/ml; and heparin, 10 mg/ml. The incubation was continued for three different time periods, two (2), eight (8), and 24 hours. Positive and negative controls were set up simultaneously. Observations, for example of cell morphology, growth, and viability, were recorded

RNA Isolation

At each time point, cells were harvested, lysed immediately by adding 1 ml of Trisol reagent (Invitrogen, Carlsbad, Calif.), the RNA was isolated as per the manufacturer's instructions, and frozen at minus 80° C. Five μg of RNA was reverse transcribed using Superscript II RT (Invitrogen, Carlsbad, Calif.) as per the instructions to obtain cDNA. One μl of cDNA was used for PCR performed as described below

Tyrosine kinase PCR-based expression experiments can be skewed if cells used in the experiment are compromised by reagent toxicity, giving rise to mRNA yields of differing orders of magnitude. Therefore, prior to being used in the experiment, the cells were visualized microscopically for viability and for any detachment from the wells. Cells from treatments for all doses and time points were >90% viable and remained adherent to the wells.

Kinase Profiling

The kinase profiling assay was established by Robinson and colleagues (Robinson et al., Proc. Natl. Acad. Sci. USA, 93:5958, 1996; and Kung et al., J. Biomed Sci 5:74, 1998), and was used here with slight modifications. Thus, various tyrosine kinase cDNA transcripts were amplified with a cocktail of degenerate primers derived from conserved motifs DFG and DVW located within the kinase catalytic domains (Table 1).

TABLE 1

| | | |
|---|---|---|
| Primer | Sequence | Sequence I.D. |

RAGE PRIMERS-SENSE

| | | |
|---|---|---|
| 5′TYKI-14 | CCAGGTCACCAARRTWGGNGAYTTYGG | SEQ ID NO. 1 |
| 5′TYKI-15 | CCAGGTCACCAARRTIDCNGAYTTYGG | SEQ ID NO. 2 |
| 5′TYKI-16 | CCAGGTCACCAARRTTDCNGAYTTYGG | SEQ ID NO. 3 |
| 5′TYKI-17 | CCAGGTCACCAARRTIWGYGAYTTYGG | SEQ ID NO. 4 |
| 5′TYKI-18 | CCAGGTGACCAARRTTGYGGAYTTYGG | SEQ ID NO. 5 |

RAGE PRIMERS-ANTI-SENSE

| | | |
|---|---|---|
| TYKI-A3 | GACAGGTTACCRHANGMCCAAACRTC | SEQ ID NO. 6 |
| TYKI-C3 | GACAGGTTACCRHANGMCCACACRTC | SEQ ID NO. 7 |
| TYKI-G3 | GACAGGTTACCRHANGMCCAGAGRTC | SEQ ID NO. 8 |
| TYKI-T3 | CACAGGTTACCRHANGMCCATACRTC | SEQ ID NO. 9 |
| TYKI-YAG3 | CACAGGTTACCRHARCTCCAYACRTC | SEQ ID NO. 10 |
| TYKI-RAG3 | CACAGGTTACCRHARCTCCARACRTC | SEQ ID NO. 11 |
| TYKI-AT3 | CACAGGTTACCRAACATCCAKACRTC | SEQ ID NO. 12 |

Thus, the 5′ (sense) primer encoding the amino acid sequence K [V/I][S/C/G] DFG [SEQ ID NO. 13] was represented by: 5′-AAR RTT DCN GAY TTY GG [SEQ ID NO. 14]. The 3′ (antisense) primer encoding the amino acid sequence DVW [S/A][F/Y] [SEQ ID NO. 15] was represented by: 5′-RHA IGM CCA IAC RTC [SEQ ID NO. 16]. The mixed bases shown in the above sequences are as follows: (1) N=A+C+T+G; (2) D=A+T+G; (3) H=A+T+C; (4) R=A+G; (5) Y=C+T; (6) M=A+C; and (7) I=deoxyinosine. The 5′ primer was labeled with [γ-³³P]-ATP (NEN Life Science Products, Boston, Mass.) catalyzed by a T4 polynucleotide kinase (Invitrogen, Carlsbad, Calif.). The 5′ primers was end-labeled with P³³ using the T4 polynucleotide kinase (Invitrogen, Carlsbad, Calif.). The 50 μl PCR reaction contained 5 μl 10×PCR buffer (Perkin Elmer, Boston, Mass.); 2 μl MgCl2 solution; 1 μl 10 mM dNTPs; 5 μl P33 labeled 5′ primer; 1 μl 3′ primer; 1 μl sample cDNA; and 1 μl TaqGold polymerase (Perkin Elmer, Boston, Mass.). PCR was performed as follows with a low stringency element to help the binding of the degenerate primers to kinase targets. This was accomplished by the following: 94° C. for 10 min for 1 cycle, followed by 5 cycles of 94° C. for 1 min, 45° C. for 1.5 min, and 72° C. for 15 sec. This was followed by the high stringency PCR element where amplification of kinase targets was accomplished by the following: 94° C. for 1 min, 56° C. for 1.5 min and 72° C. for 20 sec plus 2 sec/cycle for 23 additional cycles.

The PCR-amplified products were electrophoresed in a 2.4% agarose gel (3:1 ratio of Nusieve GTG and agarose LE; BMA, Rockland, Me.). The 153-177 bp bands were excised from the gel and DNA was extracted using the QIAEX II® gel extraction kit (QIAGEN, Valencia, Calif.). The activity of the eluted DNA was determined by liquid scintillation counting and each sample was normalized to 20,000-cpm/μl by dilution with nuclease-free H2O.

Equivalent amounts of radioactive DNA from each sample were digested with restriction enzymes (New England Biolabs, Beverly, Mass.), after which the digested products were resolved on a 7% acrylamide gel (10:1 acrylamide/bis-acrylamide; Bio-Rad Laboratories, Hercules, Calif.). The gel was dried and subjected to autoradiography.

Detailed computerized restriction digest maps are available for over 100 tyrosine kinases for each commercially available enzyme. Thus, by examining the images on the autoradiograph, differentially expressed kinase bands were mapped for each digest. The size of each differentially expressed band was assessed based on a standard size marker ran on each gel. When a differentially expressed digest band was observed on the autoradiograph, the digestion was repeated for reproducibility. Several kinases possess more than one enzyme restriction site, where available, they were used to verify that a kinase was correctly identified.

Quantitative Real Time PCR

After certain kinases were identified as being affected by procyanidins, quantitative real time PCR was performed using GenAmp 5700 Sequence Detection System (PE Biosystems, Foster City, Calif.) to confirm the kinase identification was correct.

Briefly, optimal PCR primers were designed for each kinase using Primer Express software (PE Biosystems, Foster City, Calif.). For ErbB2, the primers were: forward primer 5′-AGGGAAAACACATCCCCCAA [SEQ ID NO. 17], and reverse primer 5′-TTGGCAATCTGCATACACCAG [SEQ ID NO. 18]. For KDR, the primers were: forward primer 5′-CTTCCAAGTGGCTAAGGGCA [SEQ ID NO. 19] and reverse primer 5′-GGCGAGCATCTCCTTTTCTG [SEQ ID NO. 20]. For MAPK11, the primers were: forward primer 5′-ACGCCCGGACATATATCC [SEQ ID NO. 21], and reverse primer 5′-GTCCAGCACCAGCATCCT [SEQ ID NO. 22]. Primers for housekeeping genes, actin or GAPDH, used as standards, were purchased from PE (Perkin Elmer, Boston, Mass.).

PCR was performed using the SYBR Green technology (S. A. Bustin, Quantification of mRNA Using Real-time Reverse Transcription PCR (RT-PCR): Trends and Problems. J. Mol. Endocrinol. 29 (2002), pp. 23-39). PCR reactions were prepared in triplicate. Each 50 μl PCR reactions contained 5 μl of optimally diluted cDNA sample, 3 μl each of optimal forward and reverse primers, 5 μl of 10×SYBR PCR buffer, 4 μl of dNTP blend (2.5 mM), 6 μl of 25 mM MgCl2, 0.25 μl of AmpliTaq Gold and 23.75 μl of H2O. Standard curves for both target and housekeeping genes were prepared from pre-prepared reference samples. The relative concentration of the target gene was determined from the standard curve and was normalized to the amount of housekeeping gene.

Cell Proliferation

To assess cell proliferation, HAEC (2.5×104 cells/15 mm wells) were cultured with Human Endothelial-Serum Free Medium 200 (Cascade Biologics Inc., Portland, Oreg.) supplemented with EGF and FGF (10 ng/ml) in the absence or presence of VEGF (10 ng/ml) as indicated (Battista P J, Bowen H J, & Gorfien S F (1994) A serum-free medium for the culture of human umbilical vein. endothelial cells, Focus 17:10-13.). Endothelial cells were treated with cocoa oligomers and controls for 24 and 48 hours. At this time, cells were pulsed with 0.6 μCi [3H]thymidine/15 mm well for 12 h, and [3H] thymidine incorporation into DNA was measured as an indicator of cell proliferation (Ferrara et al. 1991, Endocrinology 129, 896-900).

Immunoprecipitation and Immunoblotting Procedures

The purpose of these experiments is to show, at the protein level, the effects of cocoa procyanidins on the usage of certain receptors. Erb B2 receptor can be assayed in this way.

For immunoblotting analysis of ErbB2 after immunoprecipitation, cells are lysed with ice-cold lysis buffer [150 mM NaCl, 10 mM Tris-HCl (pH 7.4), 1 mM EDTA, 0.5% NP40, and 1% Triton X-100] containing 1 mM Na3VO4. For each treatment, equal amounts of protein (100 μg) are precleared by incubation with a mixture of protein A/protein G-Sepharose beads for 1 h at 4° C. After removal of the Sepharose beads by low-speed centrifugation, the supernatants are transferred to fresh tubes. Lysate supernatants are incubated in lysis buffer overnight at 4° C. in the presence of 2 μg/ml of specific antibodies. Immune complexes are collected by incubating lysates with 25 μl of protein A-Sepharose beads (50% suspension) for another 2 h at 4° C. Immunoprecipitates are then washed three times with lysis buffer and once with PBS (pH 7.4) containing 1 mM Na3VO4. The proteins are extracted with 2-fold concentrated Laemmli sample buffer [125 mM Tris-HCl (pH 6.8), 20% glycerol, 4% SDS, 10% β-mercaptoethanol, and 0.00125% bromphenol blue], boiled for 4 min, separated on 7.5% SDS-PAGE, and analyzed by Western blotting.

In Vitro Kinase Assay

The purpose of these experiments is to assay the level of phosphorylation of the isolated kinase before and after stimulating the cells.

Confluent HAEC are serum starved for 24 h, and the cells are then stimulated with VEGF or 1.0 mM H2O2 and lysed. Precleared cell lysates (450 μg proteins) are incubated overnight with anti-VEGFR-1 and 2 antibodies, and the resulting immune complexes are collected with protein A-Sepharose beads and washed. The washed beads are incubated for 1 h on ice in 15 μl of kinase buffer [100 mM Tris-HCl (pH 7.0), 0.2% β-mercaptoethanol, 20 mM MgCl2, and 0.2 mM Na3VO4] containing the specific catechin. The kinase reaction is initiated by the addition of 5 μCi of [—32P] ATP (ICN Biochemicals, Irvine, Calif.) into a final volume of 20 μl and incubated at 30° C. for 15 min. The reaction is stopped by adding Laemmli sampler buffer, and proteins are separated by electrophoresis on 7.5% SDS gels. The resulting gels are stained for 30 min in 0.1% (w/v) Coomassie Blue R-250 in 30% methanol and 10% acetic acid, and destained in the same solution without the Coomassie Blue dye. The gels are then washed extensively in this solution and exposed to Fuji X-ray film.

Effect of Procyanidins on Human Micro Dermal Endothelial Cells

ErbB2 is an important receptor for the growth of the vascular capillary cells into new tissue. These micro vascular cells express many different proteins compared to aortic endothelial cells and these microvascular cells are the cells more typically involved in tumor angiogenesis, inflammation, and wound healing. Therefore, kinase expression profiling, real time PCR, and cell proliferation experiments were conducted using human micro dermal endothelial cells (HMDEC) undergoing angiogenic (i.e., growth) stimulus induced with 1 mM of H₂O₂. The cells were stimulated for 30 min and harvested after 4 hours. The experiments were performed as described above for HAEC (but HAECs were cultured for 2, 8, and 24 hours and no H₂O₂ was used). Beta actin expression was used to normalize PCR expression results.

The cells were also tested for changes in phosphorylation. Thus, a Western blot was prepared using protein lysates of 50 μg/ml pentamer-pretreated (48 hr) and control HMDEC. The lysates were prepared as described above under “Immunoprecipitation and immunoblotting procedure.” The blot was probed with recombinant HRP-Anti-PhospoTyrosine (RC20H) antibody. Protein samples were standardized by having equal staining to anti-tubulin proteins. Additionally, Western blot using protein lysates of 50 μg/ml pentamer-pretreated and control HMDEC was prepared. Cells were exposed to 1 mM H2O2 for 30 minutes and harvested after 20 minutes. The blot was probed with recombinant HRP-Anti-PhospoTyrosine (PY20) antibody.

Results

To identify the maximum number of differentially expressed kinases, a panel of 30 different restriction enzymes was used in this study, providing the means of identifying over 150 different kinases. After treatment with procyanidin monomer, dimer, pentamer and octamer isolated and purified from cocoa, expression of the most tyrosine kinases remained unchanged. For example, when digested with RsaI, the kinases Trk, Gsk-3b and Slk appeared to be unaffected by the procyanidin treatment. These unaffected bands acted as internal controls to authenticate the efficiency of the digest and the relative level of expression between the different kinases.

The most numerous, consistent and intense differences in expression occurred at the eight-hour time point. For example, at the two-hour time point, a total of 8 differentially expressed bands with a reproducibility rate of 6 of 8 (75%) were observed with 10 μg/ml procyanidin dose. At the eight-hour time point, 20 differentially expressed bands with a reproducibility rate of 18 of 20 (90%) were observed. Ninety percent of these differentially expressed bands were observed when comparing the control treatment to the pentamer and octamer treatments. The kinases that were reproducibly differentially expressed at different doses and time points were selected for further study. ErbB2, MAPK11 and KDR kinases were studied in more detail.

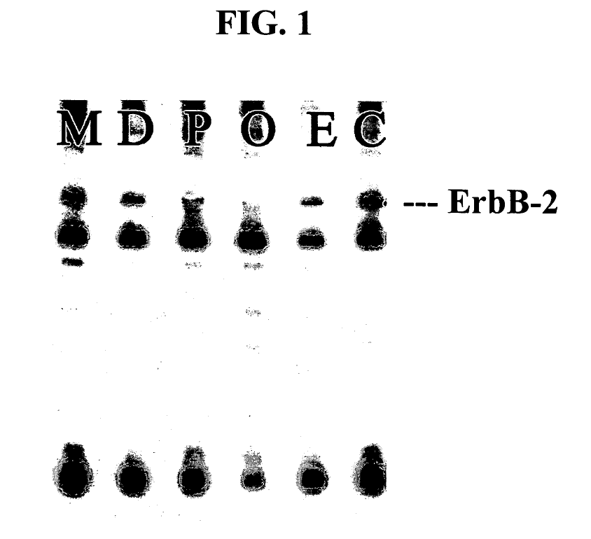

Procyanidins, particularly pentamer and octamer, down regulated the expression of ErbB2 kinase as shown by the restriction enzyme Rsa I digestion gel (FIG. 1). A similar pattern of down regulation was observed when the kinase PCR products were digested with Bsp1286 I restriction enzyme; ErbB2 contains this restriction site. The same down regulation was observed when the aortic endothelial cells were isolated from a different individual.

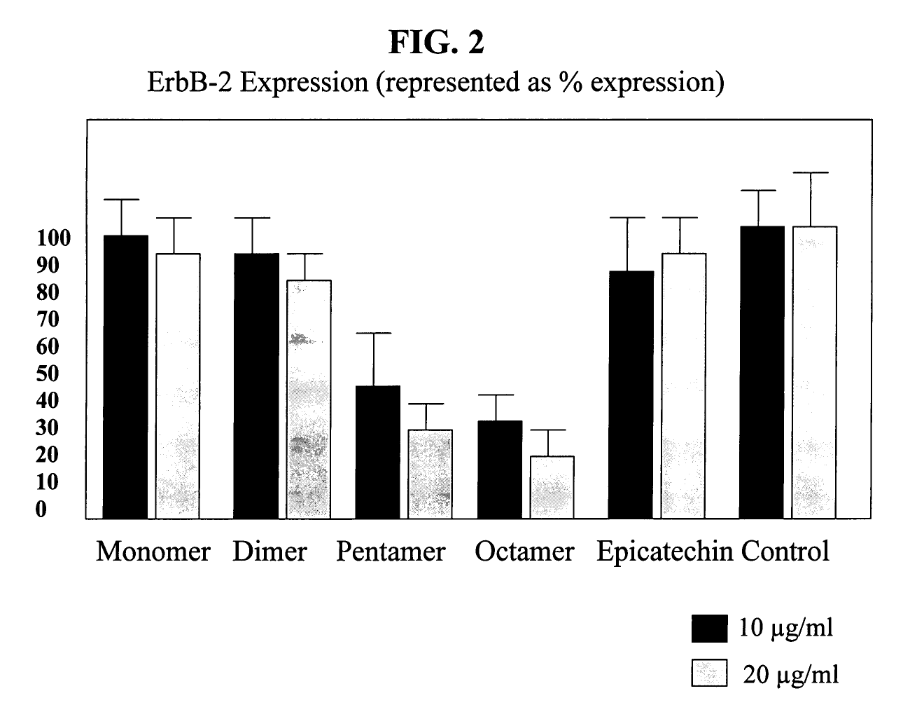

The above results were verified using quantitative real time PCR and ErbB2-specific primers. A significant decrease in expression was observed, particularly when pentamer and octamer were used at the dose of 20 μg/ml (8 hours) (FIG. 2).

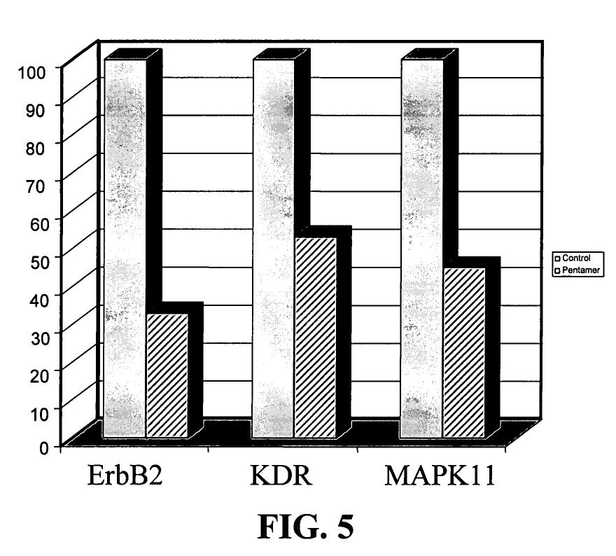

Since ErbB2 may involve expression of the VEGF (vascular endothelial growth factor) and MAPK (mitogen-activated protein kinase) system, further real time PCR experiments were conducted and revealed a decrease in VEGFR-2/KDR (vascular endothelial growth factor receptor 2/kinase insert domain receptor), the main mitogenic kinase receptor for VEGF, and MAPK11/p38beta2 kinase (FIG. 5).

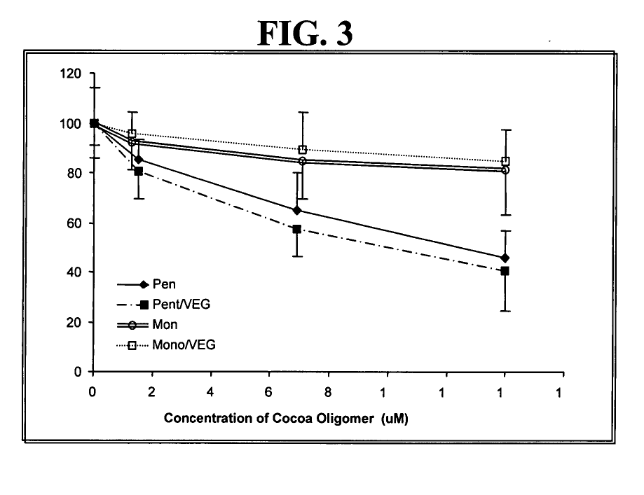

Proliferation experiments, conducted to determine whether procyanidins change growth characteristics of HAEC, showed inhibition of cell proliferation (FIG. 3). Referring to FIG. 3, the pentamer treatment, in particular, decreased cell proliferation and the cells could not be rescued by the addition of VEGF (10 ng/ml).

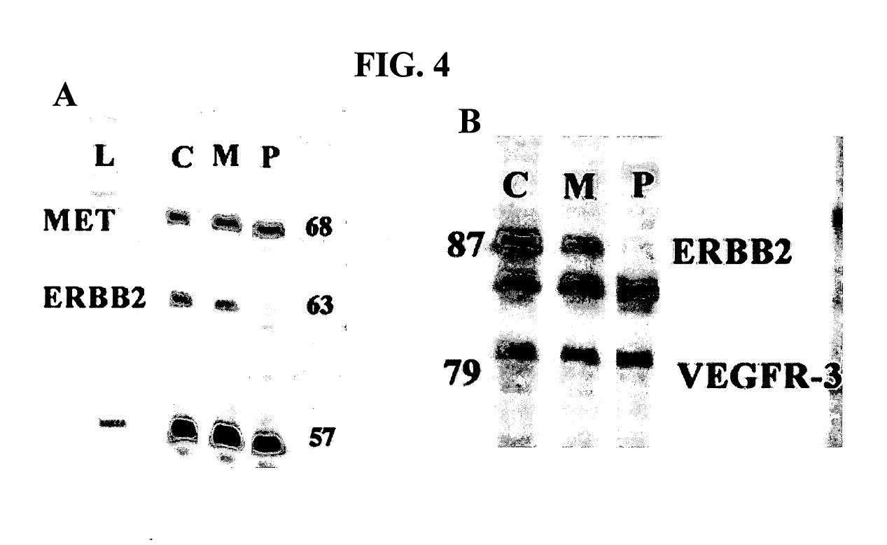

In the experiments with micro dermal epithelial cells, ErbB2 expression was down regulated with the monomer and pentamer treatments in both the RsaI (FIG. 4A) and Bsp1286I (FIG. 4B) restriction enzyme digests. Met and VEGFR-3 represent tyrosine kinases that are not affected by procyanidins. On close examination, the bands from the cocoa treated samples of VEGFR-3 and the Met appear about the same intensity as controls. In contrast, the ErbB2 band from the pentamer sample is down regulated. Quantitative real time PCR experiments verified these observations (FIG. 5, showing 20 μg/ml pentamer-pretreated and control HMDEC). Referring to FIG. 5, in addition to the reduced ErbB2 expression, KDR/VEGFR-2 and MAPK11 kinases expression was also reduced.

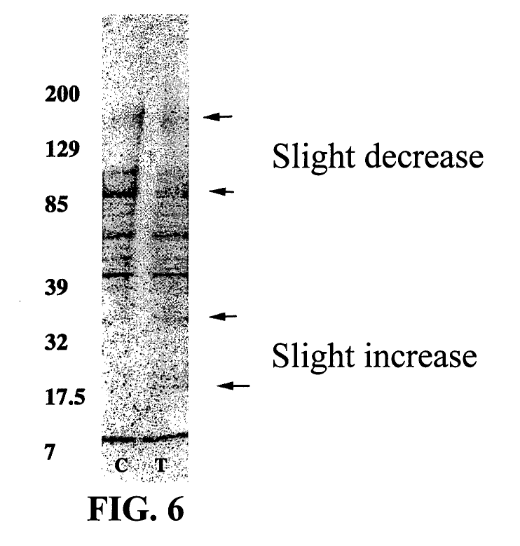

In order to determine whether procyanidins affect tyrosine kinase phosphorylation, a Western blot of HMDEC lysate was probed with RC20H antibody. Mild differences in tyrosine phosphorylation between control (C) and pentamer-treated (T) cells were observed (FIG. 6).

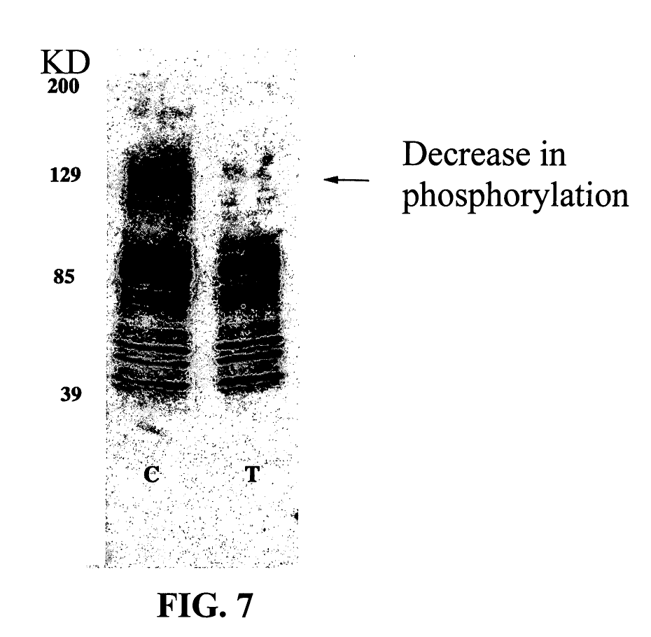

Additionally, Western blot was obtained using protein lysates of 50 μg/ml pentamer-pretreated and control HMDEC. As for the Western blot in FIG. 6, cells were exposed to 1 mM H2O2 for 30 minutes and harvested after 20 minutes. The blot was probed with recombinant HRP-Anti-PhospoTyrosine (PY20) antibody. Referring to FIG. 7, arrows indicate changes in tyrosine phosphorylation between the control (C) and procyanidin-treated cells (T). The procyanidin treated cells have reduced tyrosine phosphorylation around the 129 kDa molecular weight.

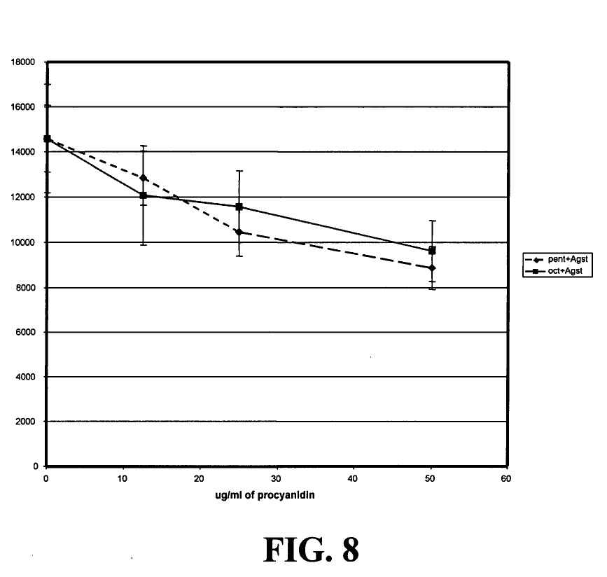

Proliferation of HMDEC treated with varying concentration of pentamer and octamer is shown in FIG. 8. Cells were exposed to 1 mM H2O2 (pro growth stimulus) for 30 minutes and harvested after 48 hours. Referring to FIG. 8, both pentamer and octamer show dose dependent inhibition of growth.

The growth of most types of tumors is dependent on angiogenesis and disruption of the angiogenic process generally causes suppression. Angiogenic process can occur by activating both vascular endothelial growth factor (VEGF) receptors and epidermal growth factor (Erb) receptor signaling. The interaction between the ErbB tyrosine kinase receptors and their ligands plays an important role in angiogenesis via the regulation of autocrine and paracrine loops. It has been reported that overexpression of the ErbB2 receptor result in induction of the basal level of VEGF and exposure to heregulin, the ligand for ErbB3 and ErbB4, further enhances VEGF secretion. Thus lowering levels of ErbB2 may reduce growth by one mechanism and also inhibit the efficiency of VEGF production by removing available ErbB2 for dimerization with activated ErbB3 and ErbB4. Since both ErbB2 and VEGF can activate the MAPK kinase system, the above observation that procyanidins down regulate MAPK11 and KDR (VEGF receptor), is consistent with the down regulation of ErbB2 and its consequent effect on growth inhibition. Thus, procyanidins can have an effect on both endothelial and tumor angiogenesis.

1. A method of treating a condition associated with overexpression of ErbB2 kinase comprising administering to a human or a veterinary animal, which overexpresses ErbB2 kinase, an effective amount of a compound selected from the group consisting of epicatechin, catechin, a polymeric compound having the formula: 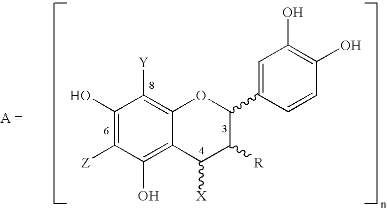
wherein
n is an integer from 2 to 18;
R and X each have either α or β stereochemistry;
R is OH;
the substituents of C-4, C-6 and C-8 are X, Z and Y, respectively, and bonding of monomeric units occurs at C-4, C-6 and C-8; and
when any C-4, C-6 or C-8 are not bonded to another monomeric unit, X, Y and Z are hydrogen,
and a pharmaceutically acceptable salt, derivative or oxidation product thereof.

2. The method of claim 1, wherein the condition associated with overexpression of ErbB2 is cancer characterized by ErbB2 kinase overexpression.

3. The method of claim 2, wherein n is 5.

4. The method of claim 2, wherein n is 8.

5. The method of claim 2, wherein the cancer is breast cancer.

6. The method of claim 2, wherein the cancer is metastatic breast cancer.

7. The method of claim 2, wherein the cancer is ovarian cancer.

8. The method of claim 2, wherein the cancer is laryngeal carcinoma.

9. The method of claim 2, wherein the cancer is prostate cancer.

10. The method of claim 2, wherein the cancer is selected from the group consisting of bladder, salivary gland, endometrial, pancreatic and non-small-lung cell carcinoma.

11. The method of claim 2, wherein the compound is administered in combination with an additional chemotherapeutic agent or to enhance the effect of chemotherapy.

12. The method of claim 3, wherein the cancer is breast cancer.

13. The method of claim 3, wherein the cancer is metastatic breast cancer.

14. The method of claim 3, wherein the cancer is ovarian cancer.

15. The method of claim 3, wherein the cancer is laryngeal carcinoma.

16. The method of claim 3, wherein the cancer is prostate cancer.

17. The method of claim 3, wherein the cancer is selected from the group consisting of bladder, salivary gland, endometrial, pancreatic and non-small-lung cell carcinoma.

18. The method of claim 3, wherein the compound is administered in combination with an additional chemotherapeutic agent or to enhance the effect of chemotherapy.

19. The method of claim 4, wherein the cancer is breast cancer.

20. The method of claim 4, wherein the cancer is metastatic breast cancer.

21. The method of claim 4, wherein the cancer is ovarian cancer.

22. The method of claim 4, wherein the cancer is laryngeal carcinoma.

23. The method of claim 4, wherein the cancer is prostate cancer.

24. The method of claim 4, wherein the cancer is selected from the group consisting of bladder, salivary gland, endometrial, pancreatic and non-small-lung cell carcinoma.

25. The method of claim 4, wherein the compound is administered in combination with an additional chemotherapeutic agent or to enhance the effect of chemotherapy.


SEQUENCE LISTING (22 sequences)

SEQ ID NO: 1 - DNA, 27 bp, Artificial Sequence
  synthetic primer
ccaggtcaccaarrtwggngayttygg

SEQ ID NO: 2 - DNA, 27 bp, Artificial Sequence
  synthetic primer
ccaggtcaccaarrtndcngayttygg

SEQ ID NO: 3 - DNA, 27 bp, Artificial Sequence
  synthetic primer
ccaggtcaccaarrttdcngayttygg

SEQ ID NO: 4 - DNA, 27 bp, Artificial Sequence
  synthetic primer
ccaggtcaccaarrtnwgygayttygg

SEQ ID NO: 5 - DNA, 27 bp, Artificial Sequence
  synthetic primer
ccaggtcaccaarrttgyggayttygg

SEQ ID NO: 6 - DNA, 26 bp, Artificial Sequence
  synthetic primer
cacaggttaccrhangmccaaacrtc

SEQ ID NO: 7 - DNA, 26 bp, Artificial Sequence
  synthetic primer
cacaggttaccrhangmccacacrtc

SEQ ID NO: 8 - DNA, 26 bp, Artificial Sequence
  synthetic primer
cacaggttaccrhangmccagacrtc

SEQ ID NO: 9 - DNA, 26 bp, Artificial Sequence
  synthetic primer
cacaggttaccrhangmccatacrtc

SEQ ID NO: 10 - DNA, 26 bp, Artificial Sequence
  synthetic primer
cacaggttaccrharctccayacrtc

SEQ ID NO: 11 - DNA, 26 bp, Artificial Sequence
  synthetic primer
cacaggttaccrharctccaracrtc

SEQ ID NO: 12 - DNA, 26 bp, Artificial Sequence
  synthetic primer
cacaggttaccraacatccakacrtc

SEQ ID NO: 13 - protein, 6 aa, Artificial Sequence
  synthetic peptide
KXXDFG

SEQ ID NO: 14 - DNA, 17 bp, Artificial Sequence
  synthetic primer
aarrttdcngayttygg

SEQ ID NO: 15 - protein, 5 aa, Artificial Sequence
  synthetic peptide
DVWXX

SEQ ID NO: 16 - DNA, 15 bp, Artificial Sequence
  synthetic primer
rhangmccanacrtc

SEQ ID NO: 17 - DNA, 20 bp, Artificial Sequence
  synthetic primer
agggaaaacacatcccccaa

SEQ ID NO: 18 - DNA, 21 bp, Artificial Sequence
  synthetic primer
ttggcaatctgcatacaccag

SEQ ID NO: 19 - DNA, 20 bp, Artificial Sequence
  synthetic primer
cttccaagtggctaagggca

SEQ ID NO: 20 - DNA, 20 bp, Artificial Sequence
  synthetic primer
ggcgagcatctccttttctg

SEQ ID NO: 21 - DNA, 18 bp, Artificial Sequence
  synthetic primer
acgcccggacatatatcc

SEQ ID NO: 22 - DNA, 18 bp, Artificial Sequence
  synthetic primer
gtccagcaccagcatcct